(19) United States
(12) Patent Application Publication
(10) Pub. No.: US 2009/0292370 A1
(43) Pub. Date: Nov. 26, 2009

(54) Device for adjusting the configuration of a modular prosthetic leg

(76) Inventors: Martin Pusch, Duderstadt (DE); Alois Goebel, Duderstadt (DE); Vladan Princ, Chrast (CZ); Manfred Kramer, Duderstadt (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/469,097

(22) Filed: May 20, 2009

(30) Foreign Application Priority Data
May 20, 2008 (DE) 10 2008 024 749.9

(51) Int. Cl. A61F 2/80; A61F 2/74

(52) U.S. Cl. 623/38; 623/27

(57) ABSTRACT

The invention relates to a device for adjusting the configuration of a modular prosthetic leg, the leg prosthesis having a foot section, a lower leg section and a knee joint, and the knee joint being disposed via a lower part on the lower leg section and an upper part being joined in an articulated manner with the lower part, having a frame and at least one fixing device disposed on the frame for fixing the foot section and the knee joint, an adjustable bracket for a thigh shaft being attached to the frame and holding a thigh shaft that is attached to the upper part of the prosthesis in its orientation with respect to the knee joint.

The invention relates to a device for adjusting the configuration of a modular prosthetic leg, the prosthetic leg having a foot section, a lower leg section and a knee joint, and the knee joint being disposed on the lower leg section via a lower part, and an upper part being joined in an articulated manner to the lower part, the device having a frame and at least one fixing device for fixing the foot section and the knee joint mounted on the frame.

Prosthetic legs, that is, prostheses which replace the knee joint, the lower leg and the foot, generally have a modular configuration and comprise a prosthetic foot, a joining element between the prosthetic foot and a joint lower part, the joint lower part having connecting means for attaching the joining element. Moreover, a joint upper part is mounted in an articulated manner on the joint lower part, where, in addition to a monocentric mount, polycentric mounts are also possible. Disposed on the upper part of the knee joint are upper connection means, which are used for the placement of, for example, a thigh shaft. Via the thigh shaft, the other components are arranged on the remnant thigh stump by means of, for example, so-called liner technology, in some cases in connection with a vacuum pump.

If a hip disarticulation is involved, the thigh shaft is made of a joining element that forms the connection between the joint upper part and a hip joint.

The individual modules of a prosthetic leg can be oriented with respect to each other in a multitude of orientations in order to produce an individual adaptation to the user of the prosthesis. However, this multitude of positioning possibilities, in both translatory and rotational terms, results in the adjustment of the prosthetic, that is, the orientation of the individual components with respect to each other, having become more complex. By changing a multitude of parameters, the orthopaedic technician can decisively help the user of the prosthesis to tap the potential within the prosthesis as fully as possible. On the other hand, incorrect positioning can cause the prosthesis to be configured in a suboptimal manner, whereupon it may result in impairments for the user of the prosthesis.

Adjustable adapters are provided, for example, for the improvement of the prosthetic configuration with respect to the patient so that the prosthetic knee joint can be displaced within the sagittal plane in relation to the upper connecting means or the lower part. The lower leg shaft length can likewise be adjusted.

German Patent Application 10 2006 021 788 A1 describes a display system for representing ground reaction forces of a human in the adjustment of a prosthesis or orthesis configuration with a measuring plate that has at least one sensor device. The sensor signals are evaluated. A projection device displays recorded sensor data. Vertical and horizontal components of a ground reaction force on the person can be depicted here, so that it is possible to display directly how the curve of the force vector acts on the prosthesis or the stump of the prosthesis wearer. This makes it possible to explain to inform the patient directly about which effects a changed prosthesis configuration has on the force direction and, as a result, also on the stress on the stump.

In addition to an improved arrangement, the adjustment of the orthesis to the prosthesis user is made easier for the orthopaedic mechanical technician, so that, all in all, an improved configuration can be achieved. However, such an orientation must always occur already on the patient.

In addition, a stand-alone unit for the static configuration of modular prosthetic legs is known in the art and is sold under the name “L.A.S.A.R.-Assembly”. Three lasers are provided above plumb lines in the sagittal and frontal views. The assembly aid is equipped with adjustment points and fixing points for a knee joint and a foot, so that the prosthetic foot and the prosthetic knee joint can be fixed within the device and mounted with corresponding adapters according to recommended configuration values. The three-dimensional configuration of the modular prosthetic leg can be carried out in a measurable and reproducible manner; likewise, additional models for leg or trunk ortheses can be oriented on the device.

Starting from this prior art, the object of the invention was to provide an improved device for adjusting the configuration of a modular prosthetic leg with which the basic orientation of all relevant components with respect to each other is enabled for provision of a reproducible basic configuration. According to the invention, this object is achieved via a device having the features of the main claim. Advantageous embodiments and further developments are explained in the subclaims.

The device according to the invention for adjusting the configuration of a modular prosthetic leg, the leg prosthesis having a foot section, a lower leg section and a knee joint, and the knee joint being disposed via a lower part on the lower leg section and an upper part being joined in an articulated manner with the lower part, having a frame and at least one fixing device disposed on the frame for fixing the foot section and the knee joint, provides that an adjustable bracket for a thigh shaft is attached to the frame and keeps a thigh shaft, which is attached to the upper part of the prosthesis, oriented with respect to the knee joint. The device makes it possible to provide the complete prosthetic leg with a predetermined configuration without a patient having to struggle with a prosthetic leg that was only roughly adjusted according to empirically determined values of the orthopaedic mechanical technician.

The adjustable bracket makes it possible to orient the particular prosthetic components with respect to each other in such a way that they follow the scientific findings in a reproducible manner, so that, even after the initial creation of the prosthetic leg, on the one hand the user of the prosthetic leg has a secure feeling while walking and on the other hand needs to expend only a small amount of energy for walking.

Preferably, the bracket can be mounted on the frame in such a manner as to swivel within the sagittal plane, where the swiveling is preferably provided exclusively within the sagittal plan but not in another plane.

For adaptation to different body sizes, it is provided that the bracket be adjustable in the vertical direction, that is, that it can travel in the direction of the knee joint or the lower leg section or the foot section or be moved away from it. For this purpose, the bracket is preferably displaceably mounted on the frame.

A further development of the invention provides that the bracket is rigidly mounted on the frame, where the bracket is fixed in the anterior-posterior direction in relation to the position of the knee joint and the prosthetic foot or the fixing point of the prosthetic foot or the knee joint The configuration is then adapted via the length and via a change in angle in the sagittal plane, for example, in order to compensate for or take into account a thigh flexion.

A further development of the invention provides that a base plate is assigned to the frame on which light emitters, especially laser emitters, are disposed so that a laser cross is set here to which the orthopaedic mechanical technician can refer in order to create an optimum configuration.

The bracket has at its distal end a centering device for the thigh shaft in order to optimally orient the thigh shaft with respect to the knee joint.

For this purpose, it is provided that the thigh shaft be centered on the bracket in order for there to be a precise assignment of the connecting means of the thigh shaft to the upper part. The centering device can have at least one elastic arm, clamping body or a deformation body that is/are introduced into the thigh shaft. As the elastic arm, a star-like elastic plastic plate having corresponding cutouts and projections can be provided that is introduced into the thigh shaft and rests against the inner side of the thigh shaft in a self-centering manner. Similarly, correspondingly designed clamping bodies, such as balloons or foam bodies, can be provided that are centered in the distal end region of the thigh shaft on the inside.

In order to hold the thigh shaft firmly in place, the bracket is provided in a proximal section with a clamping device that clamps in the thigh shaft. The clamping is preferably done on the inside, the clamping device having an hydraulically or pneumatically actuated flexible body that can be pressed from within against the thigh shaft. For example, the flexible body is designed as an elastic hose that is fixed, in particular clamped, on a support body, and is first introduced into the thigh shaft in an uninflated or unfilled state. After reaching a predetermined position of the bracket within the thigh shaft, the flexible body is then filled until the sleeve of the flexible body has come against the inner side of the shaft and centers it in place. The flexible body does not necessarily have to have a support body, but a support body makes the defined assignment of the thigh shaft to the desired spatial orientation easier. Different flexible bodies can be provided for different shaft sizes.

The support body is preferably designed with a tubular shape on the one hand to permit easy handling due to the low weight and on the other hand to have to provide only a low volume of the required fluid in order to place the flexible body against the inner side of the thigh shaft.

Exemplary embodiments of the invention are explained in detail below in reference to the accompanying Figures. In the drawing:

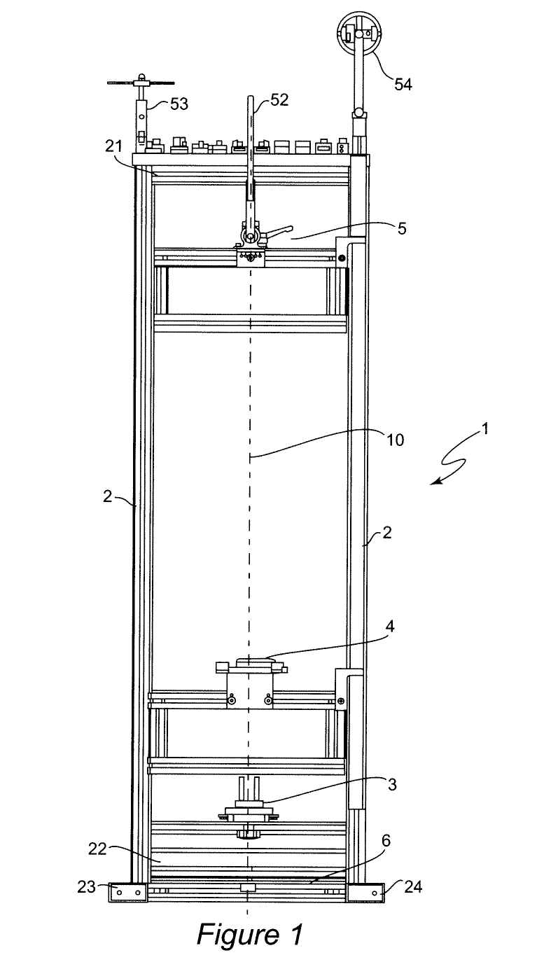

FIG. 1-shows a front view of a partially assembled device;

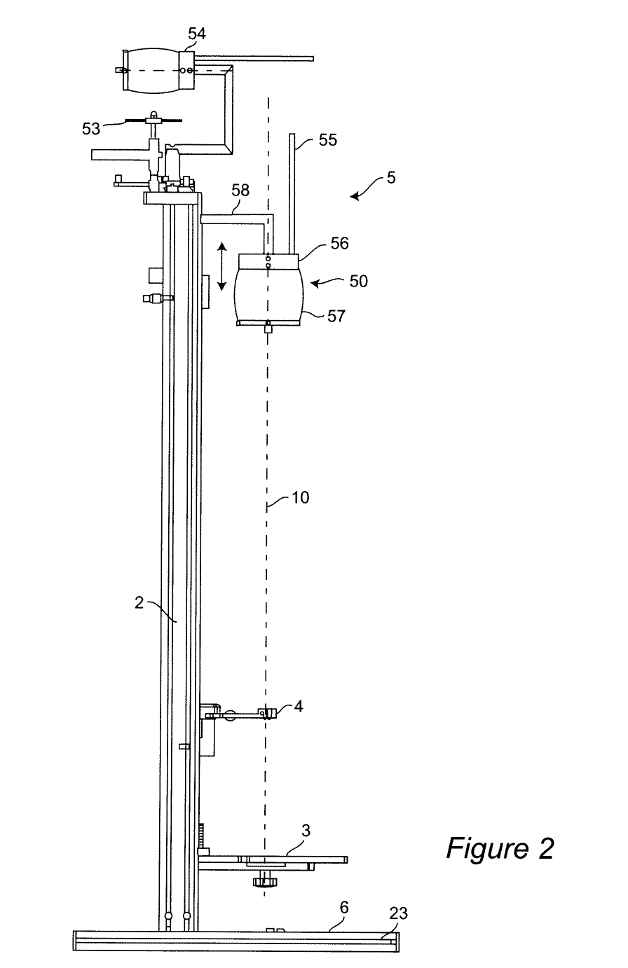

FIG. 2-shows a side view of a partially assembled device;

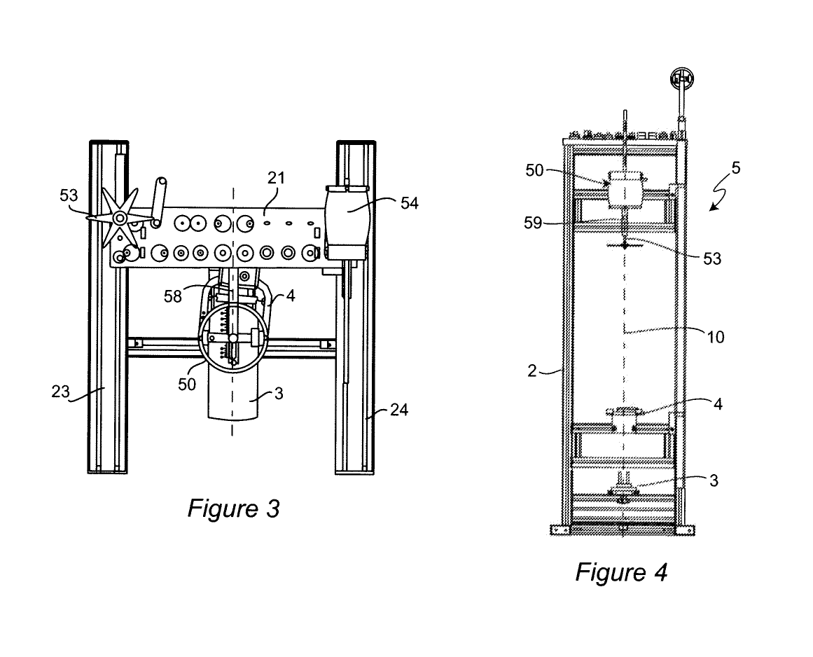

FIG. 3-shows a top view of a partially assembled device according to FIG. 2;

FIG. 4-shows a front view of a fully assembled device according to FIG. 2;

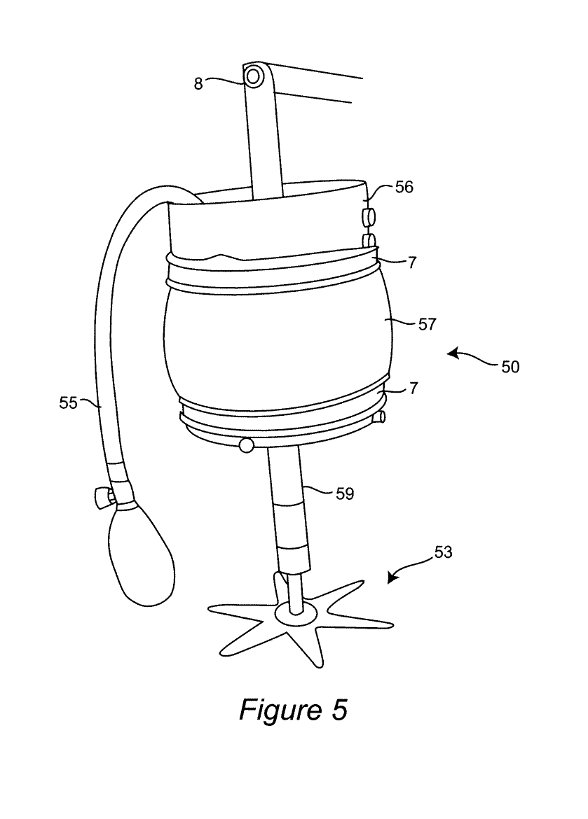

FIG. 5-shows detail of a bracket;

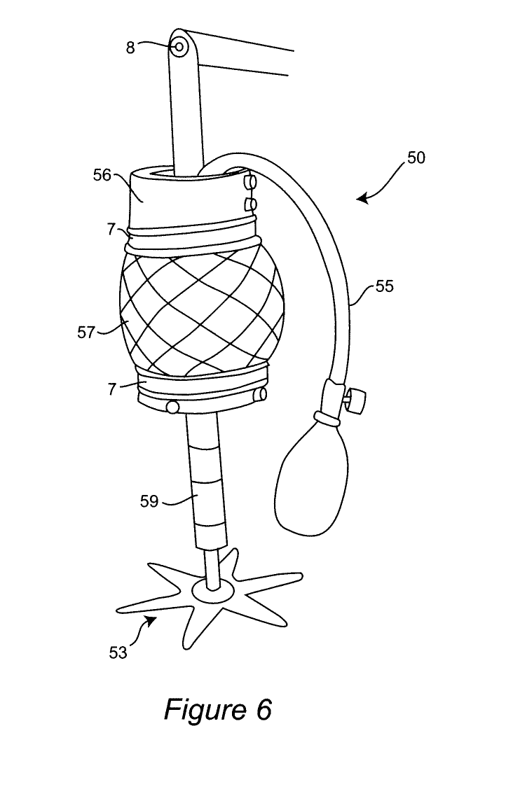

FIG. 6-shows a variant of a bracket;

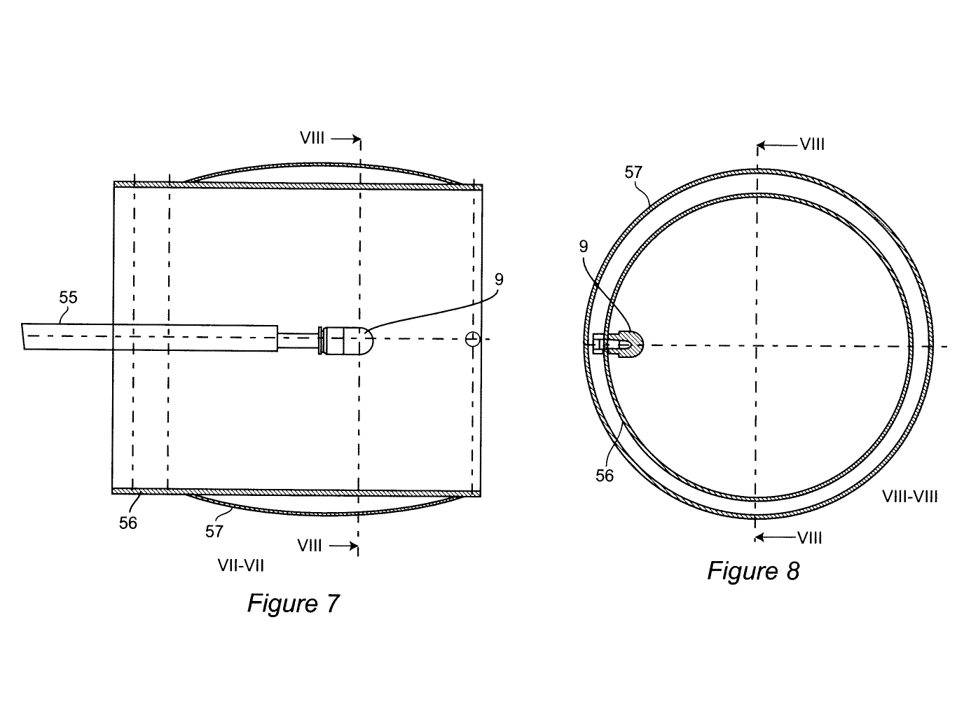

FIG. 7-shows a longitudinal sectional view of a bracket; and

FIG. 8-shows a cross-sectional view of a bracket;

FIG. 1 shows a partially assembled bracket 1 having a frame 2 made of two essentially vertically oriented side posts, which are joined to each other at the upper end and at the lower end via cross beams 21, 22. Within frame 2, a fixing device 3 for a prosthetic foot and a fixing device 4 for a lower leg shaft or a knee joint are arranged on a cross beam along center line 10. Both the prosthetic foot and the lower leg shaft are not depicted. Fixing device 3 for the prosthetic foot is disposed in the area of the lower end of device 1, and fixing device 4 for the lower leg shaft is depicted above it. In the illustrated exemplary embodiment, fixing device 4 for the lower leg shaft is designed as a clamp that can be displaced in the vertical direction along the side frame rails. In this context, fixing device 4 is always guided along center axis 10.

Fixing device 3 for the prosthetic foot is also disposed on center axis 10. Fixing device 3 is not displaceably mounted on frame 2.

A bracket 5 for a thigh shaft (also not shown) is disposed above fixing device 4 for the lower leg and fastened to frame 2. Bracket 5 is partially assembled and has a cross beam 51, which is adjustable in the vertical direction along the frame rails to enable adaptation to different body sizes and prostheses lengths. The bracket has a swiveling arm 52, which in the illustrated exemplary embodiment is disposed in the upward folded position Mounted on upper cross beam 21 are various attachments that can be installed on bracket 5, such as a centering device 53 in the form of an elastic disk and a replacement clamping device 54, whose configuration is described further below in detail. Arranged at the lower end of device 1 are a base plate 6 and stand slats 23, 24 to ensure the device's stability.

Depicted in FIG. 2 is a side view of a partially assembled device 1. In the view, one of the essentially horizontally disposed stand slats 23 can be recognized. Bracket 5 is fully assembled on centering device 53 and has a clamping device 50 with a supply device 55 for compressed air or an hydraulic fluid. A support body 56 having a flexible body 57 fastened to the outside is disposed on angle bracket 58. Angle bracket 58 is fastened to cross beam 51, which is mounted on the side posts of frame 2 in such a way as to be displaceable in the direction of the double arrow. Centering device 53 can still be arranged on support body 56, centering device 53 being arranged on the distal end of support body 56, that is, in the direction of fixing device 4 for the lower leg shaft.

Arranged on upper cross beam 21 is another replacement clamping device 54 having a support body and a flexible body, which has a smaller diameter than the clamping device 50 that is mounted in bracket 5, in order to provide an improved adaptation to the thigh shaft to be oriented.

FIG. 3 shows a top view of the device according to FIG. 2 having the two stand slats 23, 24, the centering device 53 as a star-shaped element and a replacement bracket 54. Mounted bracket 5 is mounted via angle bracket 58 above fixing device 4 for the lower leg and fixing device 3 for the prosthetic foot. Additional adapters or components can be mounted on top cross beam 21.

FIG. 4 shows a front view of the device according to FIG. 2 having additionally mounted centering device 53 below clamping device 50. The axial distance between clamping device 50 and centering device 53 is adjusted via a spindle 59.

Illustrated in detail in FIG. 5 is clamping device 50 having a distally mounted centering device 53 and spindle 59. Clamping device 50 has a support body 56, which is designed as a tube on whose exterior side a flexible body 57 is held via hose clamps 7 in such a manner that it is sealed airtight. Supply device 55 for supplying compressed air with a pressure bellows and a pressure relief valve is fastened to the inner side of support body 56 and has a fluidic connection with flexible body 57. Flexible body 50 can swivel about a joint 8 within the sagittal plane and lock in the desired angular position in order to adjust the desired configuration of a prosthesis. For the correct adjustment of the prosthetic leg, the particular modular components are put together and held in the fixing devices 3, 4, 5. A thigh shaft is mounted on a joint upper part of the prosthesis and centered via centering device 53. Centering device 53 is designed as an elastic clamping body in the shape of a star so that the projections elastically rest against the inner contour of the thigh shaft (not depicted) when a compressive force is applied. Thanks to the consistent configuration of the projections of the centering device 53, spindle 59 is kept centered.

To hold the thigh shaft securely on frame 2, clamping device 50 is introduced in the thigh shaft and blown up until flexible body 57 rests firmly against the inner contour of the thigh shaft. Angle bracket 58 is only vertically displaceable, a displacement in the transversal plane is not possible, only a tilting about the joint axis of joint 8 in the sagittal plane is possible in order to be able to optimally adjust the configuration of the prosthesis (not depicted).

FIG. 6 shows a variant of FIG. 5 having a modified spindle 59, which in the present case is designed to be longer, and a narrower clamping device 50 for a narrower prosthetic shaft.

FIG. 7 shows clamping device 50 having the tubular support body 56, the flexible body 57 fastened thereto in the form of a rubber hose and supply device 55 having an angle piece 9 mounted on the inside of support body 56 through which the compressed air is introduced from the pressure bellows in the intermediate space between support body 56 and flexible body 57. The placement of angle piece 9 is recognizable in FIG. 8, which represents a cross-sectional view of FIG. 7. If air is introduced into the intermediate space between flexible body 57 and support body 56, flexible body 57 expands until it rests against the inner contour of the prosthetic shaft and holds the prosthetic shaft there. Instead of the depicted clamps, the flexible body 57 can also be glued, welded or vulcanized to the support body. Instead of compressed air, a fluid can also be introduced into the intermediate space.

A base plate 6 on which light emitters, in particular laser emitters, are mounted can be mounted on frame 2 in order to assemble a laser cross from light bars with respect to which the prosthesis can be oriented. The laser cross intersects at center line 10, which is vertically oriented and forms the central axis.

1. A device for adjusting the configuration of a modular prosthetic leg, the leg prosthesis having a foot section, a lower leg section and a knee joint, and the knee joint being disposed via a lower part on the lower leg section and an upper part being joined in an articulated manner with the lower part, having a frame (2) and at least one fixing device disposed on the frame (2) for fixing the foot section and the knee joint, wherein an adjustable bracket (5) for a thigh shaft is attached to the frame (2) and holds a thigh shaft that is attached to the upper part of the prosthesis in its orientation with respect to the knee joint.

2. The device as described in claim 1, wherein the bracket (5) is mounted on the frame (2) in such a manner as to swivel within the sagittal plane.

3. The device as described in claim 1, wherein the bracket (5) is mounted in the frame (2) in such a manner as to be adjustable in the vertical direction.

4. The device as described in claim 1, wherein the bracket (5) is mounted in the frame (2) in such a manner as to be rigid in the anterior-posterior direction.

5. The device as described in claim 1, wherein a base plate (6) is assigned to the frame (2) on which light emitters, in particular laser emitters, are mounted that are oriented in the sagittal and frontal planes.

6. The device as described in claim 1, wherein the bracket (5) has at its distal end a centering device (53) for the thigh shaft.

7. The device as described in claim 6, wherein the centering device (53) has at least one elastic arm, clamping body and/or deformation body.

8. The device as described in claim 1, wherein the bracket (5) has in a proximal section a clamping device (50) for clamping the thigh shaft.

9. The device as described in claim 8, wherein the clamping device (50) has an hydraulically or pneumatically actuated flexible body (57) that can be pressed from within against the thigh shaft.

10. The device as described in claim 9, wherein the flexible body (57) is designed as an elastic hose that is affixed, in particular clamped, to a support body (56).

11. The device as described in claim 10, wherein the support body (56) is tubular in shape.
